(19) United States
(12) Patent Application Publication
(10) Pub. No.: US 2021/0389243 A1
(43) Pub. Date: Dec. 16, 2021

(54) SYSTEM AND METHOD FOR QUANTIFICATION OF BACTERIA IN WATER USING FLUORESCENCE SPECTRA MEASUREMENTS AND MACHINE-LEARNING

(71) Applicant: THE STATE OF ISRAEL, MINISTRY OF AGRICULTURE & RURAL DEVELOPMENT, AGRICULTURAL RESEARCH ORGANIZATION, Rishon Lezion (IL)

(72) Inventors: Shlomo SELA, Modi'in-Macabim-Re'ut (IL); Amir NAKAR, Hadera (IL); Michael BORISOVER, Herzliya (IL); Ze'ev SCHMILOVITCH, Yehud (IL)

(21) Appl. No.: 17/283,136

(22) PCT Filed: Oct. 7, 2019
(86) PCT No.: PCT/IL2019/051094

§ 371 Date: Apr. 6, 2021

(30) Foreign Application Priority Data
Oct. 11, 2018 (IL) 262298

(51) Int. Cl. G01N 21/64; G01N 1/10; G06N 20/00

(52) U.S. Cl. CPC G01N 21/64; G06N 20/00; G01N 1/10

(57) ABSTRACT

The present invention provides a system and a method for rapid quantification of bacteria in high-quality water using fluorescence spectra measurements and machine- learning. The invention is applicable to drinking water distribution systems, water purification plants, food and beverage industry, and pharma and medical industry.

### FIELD OF THE INVENTION

The invention relates to a system and a method for rapid quantification of bacteria in high-quality water using fluorescence spectra measurements and machine-learning. The invention is applicable to drinking water distribution systems, water purification plants, food and beverage industry, and pharma and medical industry.

### BACKGROUND OF THE INVENTION

Information regarding the microbiological quality of water is highly important in several fields including drinking water distribution systems, water purification plants, food and beverage industry as well as pharma and medical industries. The main importance of water microbial quality is related to public health, but it is also crucial for water managing systems in various industries.

Current methods to quantify bacteria in high-quality water require microbiology-based techniques, which are laborious, expensive and time consuming. One such method is the heterotrophic plate count (HPC) method that quantifies aerobic mesophilic bacteria. Water samples, usually 1 milliliter, are either mixed with molten nutrient-containing agar (pour plate technique) or spread on the surface of the agar (spread plate technique) and the plates are incubated for 2-5 days depending on the incubation temperature. An alternative standard method, membrane filtration, involves passing of 100-milliliter samples through a 0.45 mm filter, which is then placed on agar plate.

Given the high variability of water systems and the dynamic characteristics of water quality, data obtained at a certain time-point regarding bacterial count may not be relevant, unless it reflects an ongoing deterioration in the microbial quality of the water.

Since the drawback of the microbiological-based methods is the long-time required obtaining data on bacterial counts, several methods, which target specific bacterial pathogens, have been developed, using techniques, such as PCR and immunologic detection of antigens. These methods significantly shorten the time required to detect specific bacterial pathogens in water systems to several hours. However, these methods require professional technicians, expensive reagents and kits for each water sample, and do not provide the general data regarding bacterial counts in water, such as HPC, which are essential for water management. Moreover, these methods utilize grab samples and do not provide real-time or near real-time monitoring required for effective managing of water systems. Consequently, there is a need for rapid method to enumerate total bacterial count in water, which potentially enable real-time or near real-time databased management.

Distilled water lacks fluorescence in UV and visible range, yet natural and processed water contain bacteria, suspended and soluble organic and inorganic materials, some of which are fluorescent in this range. Microorganisms possess intrinsic fluorescence properties associated at least with certain fluorescent components present in proteins, such as the aromatic amino acids tryptophan, tyrosine and phenylalanine. Measuring excitation-emission matrices (EEM) or maps of fluorescence of water samples provides a way to detect presence of various fluorophores in water that are distinguishable based on a specific localization in the fluorescence map.

Various mathematical methods are used for analysis of EEM and extracting information on presence of aquatic fluorophores, e.g., parallel factor analysis (PARAFAC) and self-organizing maps (SOM). However, high quality water may display very weak fluorescence that is indistinguishable from the background noise, to an extent where no presence of fluorophores is detected. Consequently, there is a need for a sensitive and rapid method that will be able to isolate weak fluorescence signals at the strong noise background and generate results in real time.

It is therefore a purpose of the present invention to provide a system and a method which are capable of rapid and precise quantification of bacteria in water, ultimately in real time, thus allowing prompt technological response in case of water quality deterioration. Indeed, the invention may be incorporated on-line to various water plants and water distribution systems.

It is another objective of the invention to provide solutions which do not require laborious microbiology-based techniques, which are expensive and time consuming. The method requires no sample preparation, is non-destructive, and yields results in just a few minutes.

Further purposes and advantages of this invention will appear as the description proceeds.

### SUMMARY OF THE INVENTION

The present invention provides a method for quantification of bacteria in water comprising: obtaining a water sample; generating an excitation-emission matrix (EEM) for said water sample; and determining the concentration of bacteria in said water sample by correlating said EEM with calibrated data.

According to some embodiments, the calibrated data is obtained by determining the EEM of a plurality of test samples having known bacterial concentrations. The EEM is generated by scanning excitation wavelengths from 200 to 800 nm in 1-5 nm steps, and detecting the emitted fluorescence in 1-5 nm steps between 200 and 800 nm. In some embodiments the EEM is generated by scanning excitation wavelengths from 220 to 400 nm in 5 nm steps, and detecting the emitted fluorescence in 2 nm steps between 220 and 410 nm

According to one embodiment, the present invention provides a system for quantification of bacteria in water, the system comprising: device for generating an excitation-emission matrix (EEM) of a water sample; and logic circuity suitable for correlating said EEM with the bacterial concentration of said water sample. The logic circuitry comprises or is associated with data managing and processing apparatus. In some embodiments, the processing apparatus is a machine-learning model trained using a set of historical data.

In some embodiments, the method and system are applicable for monitoring water quality in drinking water distribution systems, water purification plants, in the food and beverage industry or pharma and medical industry. In some embodiments, the system may be incorporated into an online monitoring system.

### BRIEF DESCRIPTION OF THE DRAWINGS

FIG. 1 shows the coefficient heat map displaying the multiplication coefficient of each excitation-emission pair for the prediction formula.

FIG. 2 illustrates the correlation between the predicted and the real E. coli concentration of double-distilled water of a training set of samples (left) and validated set of samples (right).

FIG. 3 illustrates the correlation between the predicted and the real bacterial concentration of drinking water of a training set of samples (left) and validated set of samples (right).

### DETAILED DESCRIPTION OF EMBODIMENTS OF THE INVENTION

The present system and method usefully provide a mathematical modeling approach that utilizes water fluorescence measurements to extract data related to the total number of bacteria in water. The data are processed using algorithms based on methods, such as Partial Least Squares Regression (PLSR), which through machine-learning can analyze complex excitation-emission matrix (EEM) data and correlate these data to the number of bacteria in a high quality water sample.

It was presently found that measuring excitation-emission matrices (EEM) or maps of fluorescence intensities of water samples provide a way to rapidly and precisely quantify bacteria in high-quality water samples. Specifically, the linear regression between fluorescence intensities and the real bacterial concentration (as CFUs/ml) of a training set of samples (here, approximately 80% of the samples), was used for obtaining a prediction formula. This prediction formula is then be used for determining the bacterial concentration in water samples. The model is tested using a validation set of samples (here, approximately 20% of the samples). Two studies were conducted: 1) determination of E. coli concentration in double-distilled water; and 2) determination of bacterial density in natural ground water serving as drinking water.

In a first aspect, the invention is directed to a method for quantification of bacteria in water comprising: obtaining a water sample; generating an excitation-emission matrix (EEM) for said water sample; and determining the concentration of bacteria in said water sample by correlating said EEM with calibrated data.

In some embodiments the calibrated data is obtained by determining the EEM of a plurality of test samples having known bacterial concentrations. The bacterial concentrations may be determined using the heterotrophic plate count (HPC) method which quantifies aerobic mesophilic bacteria.

The water samples are scanned by spectrofluorophotometer using excitation wavelengths at a range of 200-800 nm, and an emission spectrum at each excitation wavelength at a range of 200-800 nm. Intensities at each excitation/emission wavelength are divided by the Raman scatter intensity of pure non-fluorescent water in order to normalize the data and minimize effects of machine/lamp instability.

In some embodiments, the EEM is generated by scanning excitation wavelengths from 200 to 800 nm in 1-5 nm steps, and detecting the emitted fluorescence in 1-5 nm steps between 200 and 800 nm. According to some embodiments, the EEM is generated by scanning excitation wavelengths from 220 to 400 nm in 5 nm steps, and detecting the emitted fluorescence in 2 nm steps between 220 and 410 nm.

Another embodiment relates to a system for quantification of bacteria in water, the system comprises: device for generating an excitation-emission matrix (EEM) of a water sample; and logic circuity suitable for correlating said EEM with the bacterial concentration of said water sample.

According to some embodiments, the logic circuitry comprises or is associated with data managing and processing apparatus. In some further embodiments the apparatus is a machine-learning model trained using a set of historical data.

In some embodiments, the method and system may be applied for monitoring water quality in drinking water distribution systems, water purification plants, food and beverage industry or pharma and medical industries.

The invention provides a system and method for real-time monitoring of bacterial concentration enabling effective water management for assuring the quality and safety of water. According to some embodiments the system may be incorporated into online monitoring systems in water plants or water distribution systems thereby providing accurate assessment of the microbiological quality of water.

The invention will be further described and illustrated in the following examples.

### EXAMPLES

### Example 1

Finding Prediction Formula for Determining Bacterial Concentration

Water samples, containing known bacterial concentrations (counts) were scanned by spectrofluorophotometer (Shimadzu, RF-5301PC, Kyoto, Japan) using excitation wavelengths range of 220-400 nm, and the emission spectrum at each excitation wavelength is obtained at the 220-410 nm range for generating an excitation-emission matrix (EEM). Intensities at each excitation/emission wavelength were divided by the Raman scatter intensity of pure non-fluorescent water in order to normalize the data and minimize effects of machine/lamp instability.

Principal Component Analysis (PCA; JMP Pro 13, SAS Institute, Cary, USA) is used to exclude major outliers (usually, <5% of samples). The samples are assigned their known bacterial concentration, described as the dependent variable, while the fluorescence intensities at each of the excitation/emission wavelength pairs are defined as the independent variables, thus producing 3775 different independent variables. The samples are then randomly divided into training (80%) and validation (20%) sets. This division means that the prediction formula will be based on 80% of the data, and tested on the remaining 20% of the data in order to evaluate the model's quality. A JMP Partial Least Squares (PLS) algorithm is used to obtain a prediction loadings formula, which is a list of weights given to fluorescence intensity at each combination of excitation/emission wavelength (i.e., the independent variable). This formula is applied to the validation dataset to predict bacterial counts (i.e. dependent variable). In order to test the model's prediction quality, a linear regression between the actual and predicted values is calculated and characterized with R² and Root Mean Square Error as measures of correlation and deviation respectively.

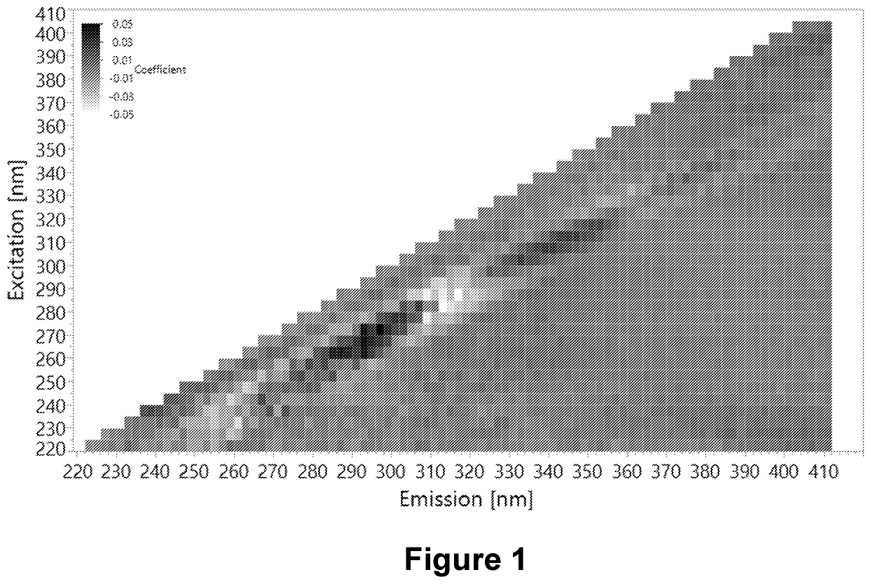

FIG. 1 shows the coefficient heat map displaying the multiplication coefficient of each excitation-emission pair for the prediction formula.

The prediction formula is calculated as a sum of all fluorescence intensities of each excitation-emission pair (X) multiplied by their individual coefficient (a) and an error (b), as shown in the following formula:

$$\hat{Y} = {\sum }_{i = 220 / 222}^{400 / 410} {a}_{i} * {X}_{i} + b$$

### Example 2

Determination of E. Coli Concentration in Double-Distilled Water

In order to initially test the ability to rapidly and precisely quantify bacteria in water samples by measuring excitation-emission matrices (EEM), 54 samples containing increasing concentrations (0-10⁸ CFU/ml) of Escherichia coli in double distilled (non-fluorescent) water were prepared. Dataset containing bacterial concentrations and their cognate EEMs in 43 water samples (80%) were used as a training set, as described in Example 1. Each bacterial concentration was done in 5 replications. The derived model was used to predict the number of bacteria in the 11 samples (20% validation set).

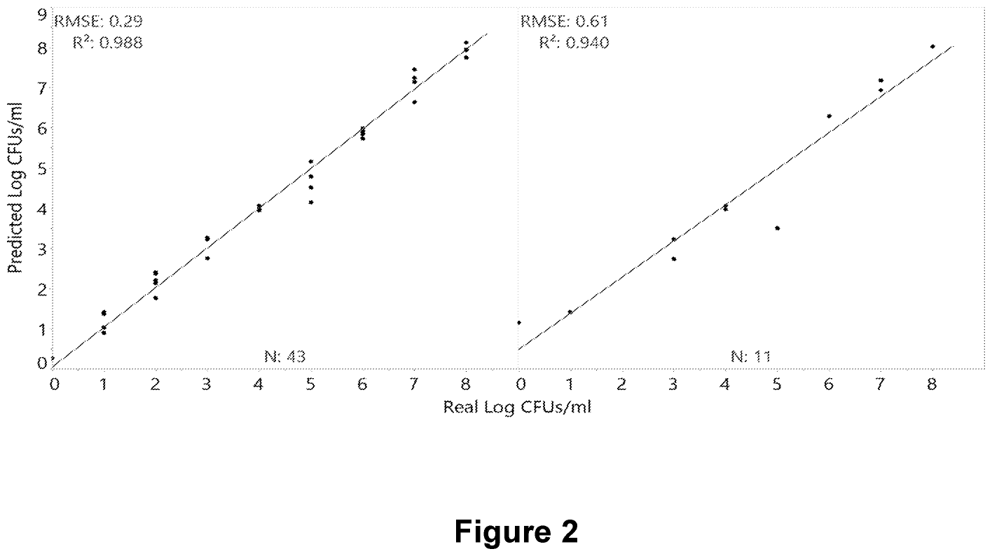

FIG. 2 shows the correlation between the predicted bacterial concentration and the real concentration. The left panel shows the training set of 43 samples (the model) and the right panel shows the validated set of 11 samples.

The model was able to detect the number of the E. coli cells at concentrations as low as 100 CFU/ml in the validation data. It correctly predicted bacterial concentration with a root mean square error (RMSE) of 0.61 log₁₀ CFU per ml and accounted for more than 90% of the variation.

### Example 3

Determination of Bacterial Density in Natural Ground Water Serving as Drinking Water

Applying of the method of the invention in critical control points of local and national drinking water distribution systems will enable real-time monitoring of the microbial quality of the water and consequently will allow prompt response in case of temporal deterioration of water quality.

In order to examine the validity of the method, 69 samples of drinking water (ground water) of known microbiological content (HPC determined by international standard methods) were read by spectrofluorometer to obtain EEM, as described in Example 1. The water samples were divided into two groups, containing 51 samples for training (74%) and 18 samples as a validation set (26%). Modeling was performed on the data from the 51 samples and the bacterial counts (HPC) in the 18 validation samples were determined.

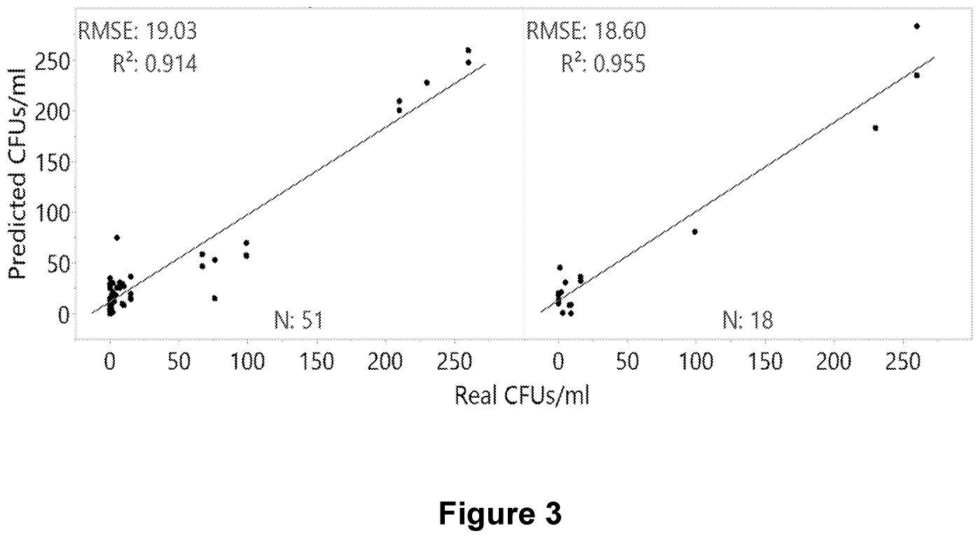

FIG. 3 shows the correlation between the predicted bacterial concentration and the real concentration of data obtained from the training set of 51 samples (left panel) and validated set of 18 samples (right panel).

The model enabled enumeration of HPC at concentrations as low as 200 CFU/ml in the overall data. The RMSE of the predicted bacterial concentration was 20 CFU/ml and the model accounted for more than 90% of the variation.

Although embodiments of the invention have been described by way of illustration, it will be understood that the invention may be carried out with many variations, modifications, and adaptations, without exceeding the scope of the claims.

1. A method for quantifying bacteria in water comprising:
i) obtaining a water sample;
ii) generating an excitation-emission matrix (EEM) for said water sample; and
iii) determining the concentration of bacteria in said water sample by correlating said EEM with calibrated data.

2. The method according to claim 1, wherein the calibrated data is obtained by determining the EEM of a plurality of test samples having known bacterial concentrations.

3. The method according to claim 1 or 2, wherein the EEM is generated by scanning excitation wavelengths from 200 to 800 nm in 1-5 nm steps, and detecting the emitted fluorescence in 1-5 nm steps between 200 and 800 nm.

4. The method according to claim 3, wherein the EEM is generated by scanning excitation wavelengths from 220 to 410 nm in 5 nm steps, and detecting the emitted fluorescence in 2 nm steps between 220 and 410 nm

5. The method according to claim 1, for monitoring water quality in drinking water distribution systems, water purification plants, food and beverage industry or pharma and medical industry.

6. A system for quantification of bacteria in water, the system comprising:
a) device for generating an excitation-emission matrix (EEM) of a water sample; and
b) logic circuity suitable for correlating said EEM with the bacterial concentration of said water sample.

7. The system according to claim 6, wherein the logic circuitry comprises or is associated with data managing and processing apparatus.

8. The system according to claim 7, wherein the processing apparatus is a machine-learning model trained using a set of historical data.

9. The system according to claim 6, for monitoring water quality in drinking water distribution systems, water purification plants, food and beverage industry or pharma and medical industry.

10. The system according to claim 6, for incorporation into an online monitoring system.
